(19) United States
(12) Patent Application Publication
(10) Pub. No.: US 2009/0010381 A1
(43) Pub. Date: Jan. 8, 2009

(54) COMPUTER TOMOGRAPH AND RADIATION DETECTOR FOR DETECTING RAYS THAT ARE ELASTICALLY SCATTERED IN AN OBJECT

(75) Inventor: Jens-Peter Schlomka, Hamburg (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONIC, N.V., EINDHOVEN (NL)

(21) Appl. No.: 10/597,080

(22) PCT Filed: Jan. 4, 2005
(86) PCT No.: PCT/IB2005/050017

§ 371 Date: Sep. 25, 2008

(30) Foreign Application Priority Data
Jan. 16, 2004 (EP) 04100135.5

(51) Int. Cl. A61B 6/08; A61B 6/00

(52) U.S. Cl. 378/7; 378/19; 378/14

(57) ABSTRACT

The invention relates to a computer tomograph and to a radiation detector for detecting elastically scattered rays. The computer tomograph comprises a radiation source for radiating primary radiation through an object which is present in an examination region. The primary radiation is partly scattered in the object owing to interactions with the object. A detector comprises detector elements by which the scattered rays are detected. These detector elements lie outside the region through which the primary radiation is passed, and their effective dimensions become smaller in the direction in which the scattering angles become smaller.

The invention relates to a computer tomograph and a radiation detector for detecting elastically scattered rays. Such devices are used, for example, as X-ray in medicine and for luggage inspection in security checks in airports. An essential property is that the detected scattered rays render it possible to draw conclusions on the material by which the rays were scattered.

A computer tomograph is known from EP 1127546 in which an X-ray source generates a fan-shaped beam of X-rays passing through an object and detected by an X-ray detector. The X-ray detector detects primary radiation with one portion of its measuring surface and scattered radiation with another portion. A collimator arrangement with a plurality of lamellae lying in planes that subdivide the fan of rays into a number of sections is present between the object and the X-ray detector, so that detector elements present in a slot parallel to the axis of rotation are hit only by radiation from the same section. Detectors are furthermore known with which in addition the energy of the detected scattered X-ray can be measured, rendering possible the use of X-ray sources which generate polychromatic X-rays.

It is an object of the present invention to improve computer tomographs and radiation detectors for the detection of elastically scattered rays.

This object is achieved, according to claim 1, by means of a computer tomograph for detecting rays that are elastically scattered in an object, wherein the object is present in an examination region and the scattered rays are scattered at different scattering angles, with

a radiation source for permeating the examination region with primary radiation, and

a detector with detector elements which lie outside the region permeated by primary radiation and whose effective dimensions become smaller in the direction of decreasing scattering angles.

The term “computer tomograph” is to be understood not just as it is generally used, but all devices are meant here by means of which cross-sectional images or layer images of objects can be generated from projections at various angles. Among them are, for example, also C-arm X-ray devices with which images are acquired of an object from various angles, wherefrom a layer image is reconstructed by means of known CT-type reconstruction methods.

“Primary radiation” is generally understood to be radiation which issues from the radiation source and permeates the examination region, for example in the form of a thin, linear or flat, fan-type ray, possibly being attenuated by an object present in the examination region, however without changing its direction. Radiation that may be used is, for example, X-radiation, but also radiation from isotopes such as gamma radiation. When penetrating the object, the rays may be scattered through known interaction with the material of the object, i.e. they change their direction and leave the object and the examination region in a direction different from the one they had when entering the examination region. If the change in direction takes place without energy losses, it is denoted elastic scattering. This radiation with changed direction forms the scattered radiation. The angle enclosed by the linear direction of the rays and the changed direction of the scattered rays is the scattering angle. The distribution of the scattered rays over various scattering angles is dependent on the material that caused the scattering and on the energy of the rays. The scattered rays are incident on the detector elements of a detector and are detected thereby.

A quantity characterizing the scattered radiation is the so-termed momentum transfer:

$$x = \frac{E}{h \cdot c} sin ( \frac{Φ}{2} ) \qquad ( 1 )$$

where c is the velocity of light, h is Planck's constant, E is the energy of the rays, and Φ is the scattering angle. It is true for small scattering angles that sin(Φ)≈Φ, so that the accuracy with which the momentum transfer is measured is proportional to the accuracies of the two influencing quantities E and Φ. In general, the ratio of the maximum measurable accuracy Δz of a quantity to an absolute value z of this quantity gives the resolution thereof. The resolution Δx/x of the momentum transfer for small scattering angles is:

$$\frac{Δ x}{x} = {\sqrt{( \frac{Δ E}{E} )}}^{2} + {( \frac{Δ Φ}{Φ} )}^{2} \qquad ( 2 )$$

where ΔE is the accuracy of the energy determination and ΔΦ the accuracy of the determination of the scattering angle.

Computer tomographs are known which use a monochromatic radiation source, so that the energy resolution ΔE/E follows from the actual bandwidth of the energy of the emitted rays. Computer tomographs are furthermore known which use a polychromatic radiation source and an energy-resolving detector, which in that case determines the energy resolution. Given a certain resolution of the energy, the resolution of the momentum transfer can be obtained in a similar order of magnitude in that the resolution of the scattering angle ΔΦ/Φ is not appreciably worse than the resolution of the energy.

The resolution of the scattering angle is determined by various influences. For example, the primary radiation has a finite thickness perpendicularly to its direction of propagation, so that scattered radiation of the same scattering angle, but originating from different locations of scattering is detected by a detector element. The size of the detector elements has a major influence on the resolution of the scattering angle. Detector elements are known which can detect radiation only with a portion of their surface area for technical reasons, the so-called sensitive region. In this case it is not the size of the detector element but the size of the sensitive region that influences the resolution of the scattering angle. Indeed, only those dimensions are decisive for the resolution of the scattering angle which extend in the direction in which the scattering angles can change, i.e. in the direction of decreasing or increasing scattering angles. Changes in the dimensions perpendicular thereto merely influence the quantity of scattered rays of the same scattering angles that can be detected.

An effective dimension is accordingly understood to be that dimension of the sensitive region of a detector element which extends in the direction of scattering angle changes. If the sensitive region of a detector element forms a rectangular surface, for example, and one side of the surface extends in the direction of scattering angle changes, then the length of this side corresponds to the effective dimension of the detector element. These considerations are valid in particular for an accuracy ΔΦ of the scattering angle, because here the change in the scattering angle can be assumed to be perpendicular to the direction of the scattered radiation.

If the resolution of the momentum transfer is to be kept constant over the entire detector or is to be kept below a maximum value, the effective dimensions of the detector elements must lie below a value which is dependent on the scattering angle and which becomes smaller in the direction in which the scattering angles become smaller, as was explained above. This is achieved in that the effective dimensions of the detector elements are made smaller in the direction of smaller scattering angles. This condition need not apply to all detector elements, depending on the required resolution, but, for example, only for those detector elements which detect scattered rays with small scattering angles. The effective dimensions of detector elements detecting scattered rays with greater scattering angles may, for example, be the same. A resolution better than the required one is then realized with the latter elements.

The detector elements may comprise besides said detector elements also further detector elements such as, for example, detector elements that detect primary radiation. It is also possible that the computer tomograph comprises further detectors, for example a first detector for detecting the primary radiation and a second detector for detecting the scattered radiation.

If a detector is formed from detector elements of equal size or detector elements all having a sensitive region of the same size, a minimum resolution is often not safeguarded for those detector elements that detect scattered rays with small scattering angles. The further embodiment of the invention as claimed in claim 2, however, renders it possible to achieve the required resolution also at detectors whose detector elements have too great effective dimensions. This is achieved in that the absorption elements reduce the effective dimensions of the detector element by covering a portion thereof. This renders it possible, for example, to improve the resolution of existing detectors. It may not be necessary to cover all detector elements of the detector, subject to the size of the detector elements, but only those which detect scattered rays with small scattering angles.

The further embodiment of claim 3 renders possible the use of a radiation source which generates polychromatic radiation, i.e. radiation with different energies. Such a radiation source is less expensive and clearly more powerful than a monochromatic source, for example in the case of X-ray radiation. Since the resolution of the scattering angle is also dependent on the energy of the radiation, an energy-resolving detector is to be used at the same time, but the additional cost thereof is absorbed by the advantage of the higher power of the radiation source.

The further embodiment of claim 4 optimizes the use of a radiation source which generates rays in the form of a flat fan. The cited publication EP 1127546 is referred to here, where the use and effect of the lamellae are described in detail. Another further embodiment as defined in claim 5 corresponds to claim 3 of EP 1127546, to which reference is made once again for further details. This further embodiment renders it possible to detect scattered rays with the computer tomograph in a first mode of operation, and to acquire conventional computer tomography images in a second mode of operation, utilizing the entire detector.

The object is furthermore achieved by means of a detector for determining elastically scattered rays, which comprises at least one column of a plurality of energy-resolving detector elements, wherein the pitch of their centers and their dimensions increased in the direction of the column to a maximum value. The detector may comprise further detector elements in addition to the above detector elements.

The term “detector element” in the computer tomograph according to the invention and in the detector according to the invention is understood to cover also a detector element which is formed by a plurality of mutually adjoining sub-elements, which are preferably of the same size. The active region of such a detector element is then formed by the totality of the active regions of all sub-elements. The adaptation of the effective dimensions to the requirements mentioned above may then take place at least approximately by way of the number of sub-elements per detector element.

The invention will be explained in more detail below with reference to the drawings, in which:

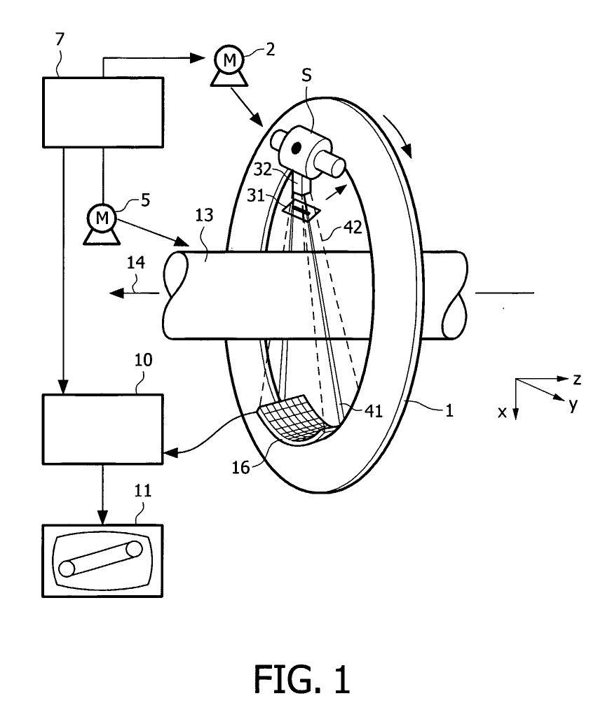

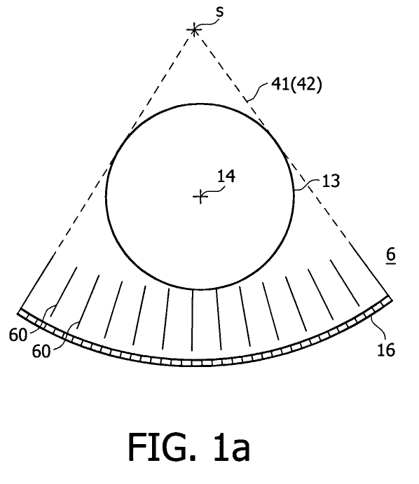

FIG. 1 diagrammatically shows a computer tomograph according to the invention,

FIG. 1a shows a collimator arrangement,

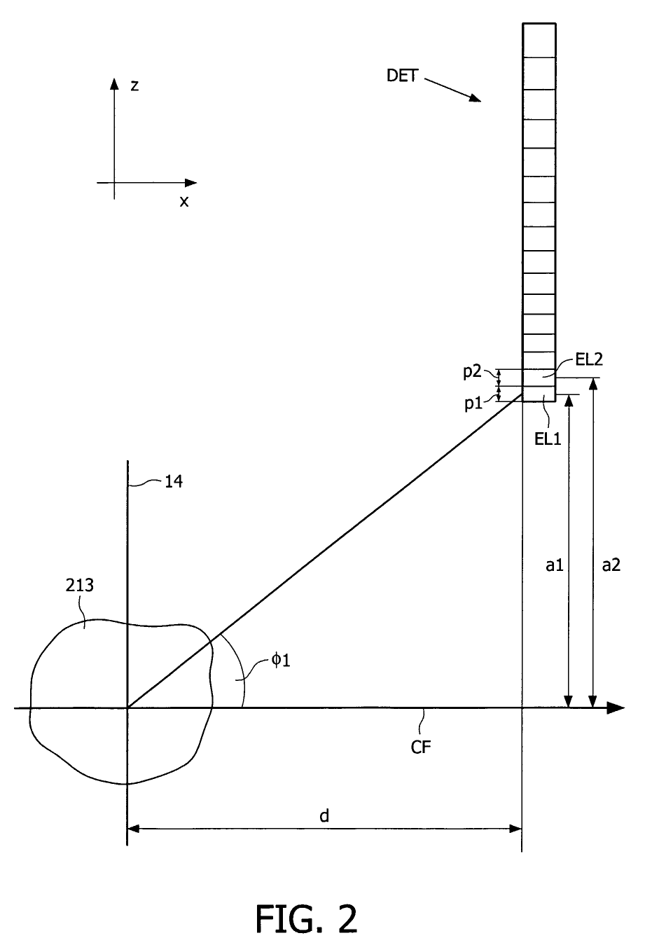

FIG. 2 shows the geometrical relationships with a first detector,

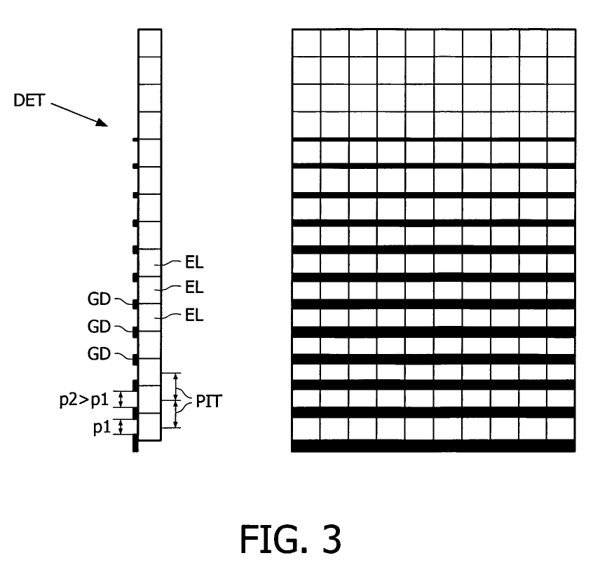

FIG. 3 shows the geometrical relationships with a second detector,

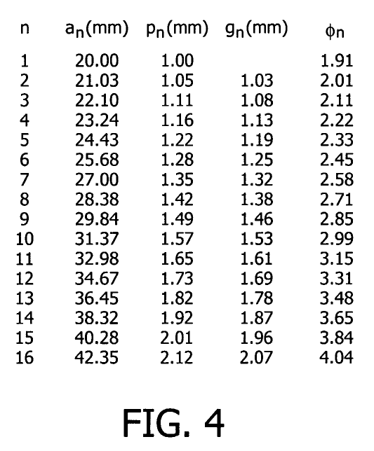

FIG. 4 lists the dimensions of a first detector,

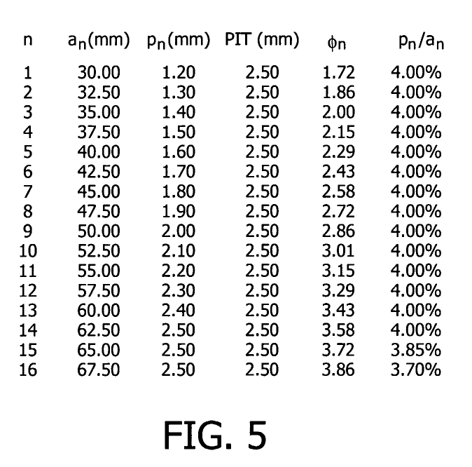

FIG. 5 lists the dimensions of a second detector, and

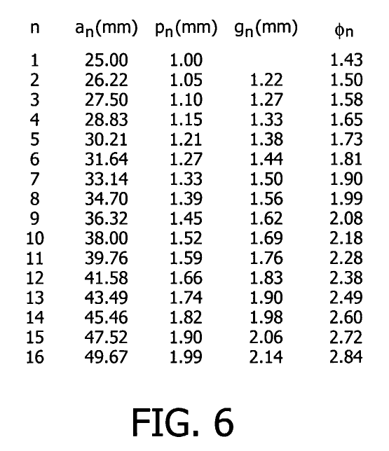

FIG. 6 lists the dimensions of a third detector.

The computer tomograph shown in FIG. 1 comprises a gantry 1 which can rotate about an axis of rotation 14. The gantry 1 is driven by a motor 2 for this purpose. A radiation source S, for example an X-ray radiator, is fastened to the gantry 1. The radiation beam used for examination is defined by a first diaphragm arrangement 31 and/or a second diaphragm arrangement 32. If the first diaphragm arrangement 31 is active, the radiation fan drawn in full lines is formed, running perpendicularly to the axis of rotation 14 which is parallel to the z-direction, having the smallest possible dimensions (for example <1 mm) in the z-direction. If the second diaphragm arrangement 32 is active in the radiation path, however, the radiation cone 42 shown in broken lines is formed, having the same shape in a plane perpendicular to the axis of rotation 14 as the radiation fan 41, but having substantially greater dimensions in the direction of the axis of rotation 14.

The radiation beam 41 or 42 passes through a cylindrical examination region 13 in which, for example, a patient is present on a patient examination table (both not shown) or alternatively a technical object. After passing through the examination region 13, the radiation beam 41 or 42 is incident on a two-dimensional detector arrangement 16 fastened to the gantry 1 and comprising a plurality of detector elements arranged in a matrix. The detector elements are arranged in rows and columns, such that the columns extend in the z-direction, i.e. parallel to the axis of rotation. The detector rows may lie in planes perpendicular to the axis of rotation, for example on a circular arc around the radiation source S. The detector rows usually contain substantially more detector elements (for example 1000) than do the detector columns (for example 16).

If the object under examination is not a patient, the object may alternatively be rotated during examination, while the radiation source S and the detector arrangement 15 are stationary. The object may also be shifted parallel to the axis of rotation 14 by means of a motor. If the motors 5 and 2 run simultaneously, a helical scanning movement of the radiation source S and the detector arrangement 16 is obtained.

In FIG. 1, the radiation beams 41 and 42, the examination region 13, and the detector arrangement 16 are mutually adapted. The dimensions of the radiation fan 41 or radiation cone 42 are chosen in a plane 14 perpendicular to the axis of rotation such that the examination region 13 is fully permeated by radiation, and the length of the rows of the detector arrangement is chosen exactly such that the radiation beams 41, 42 can be fully detected. The radiation cone 42 is chosen in accordance with the length of the detector columns such that the radiation cone can be fully caught by the detector arrangement 16. If only the radiation fan 41 passes through the examination region, it will hit the central detector row or rows.

FIG. 2 shows part of the arrangement of FIG. 1 from a different perspective. The systems of co-ordinates shown in the Figures are provided for orientation. The computer tomograph of FIG. 1 is operated in a first mode of operation. For this purpose, both the first diaphragm arrangement 31 and the second diaphragm arrangement 32 are in the radiation path between the radiation source S and the object 13, such that the fan-shaped radiation beam 41 is generated. Ideally, the radiation fan 41 has no dimension in the z-direction, so that this fan is merely shown as a line CF in FIG. 2. Furthermore, not the entire detector 16, but only a portion of a detector column DET is shown here. The detector elements of the column portion DET detect scattered radiation. It is assumed that X-rays are scattered with a scattering angle Φ 1 at the point of intersection between the axis of rotation 14 and the radiation fan. These scattered rays are incident on a detector element EL1, which is removed from the radiation fan by a distance a₁ and from the location of scattering by a distance d. Since the detector element has a dimension in the z-direction, i.e. the height pₙ, scattered rays with slightly greater and slightly smaller scattering angles can also be detected by EL1. This angular region is denoted ΔΦin equation (2). It is assumed that the sensitive region of the detector element extends over the entire height pₙ.

This is true in an analogous manner for a detector element EL2 which is removed from the radiation fan by a distance a₂. This results in a general geometrical relation valid for any detector element n:

$${Φ}_{n} = {tan}^{- 1} ( \frac{{a}_{n}}{d} ) \qquad ( 3 )$$

The portion of the elastic scattering relevant for statements on the material takes place, at least in the case of X-rays, only within a small angular region, for example between 1° and 15° in the case of X-rays having an energy between 20 and 200 keV. For greater clarification of the subsequent embodiments, the figures are not drawn true to scale. The tangent of an angle is approximately equal to the angle itself in the case of small angles, so that

$${Φ}_{n} \approx ( \frac{{a}_{n}}{d} ) \qquad ( 4 )$$

A constant distance d and a small angle Φₙ then results in:

$$\frac{Δ {Φ}_{n}}{{Φ}_{n}} \approx \frac{Δ {a}_{n}}{{a}_{n}} \qquad ( 5 )$$

That means that, as a detector element is closer to a radiation fan, the accuracy Δa must be smaller, or the dimension of the detector element in the z-direction must be smaller. The effective dimensions of the detector element are accordingly constituted by the height pₙ here. The distance or pitch between the centers of two mutually adjoining detector elements g follows from the sum of the two half heights:

$$g = {a}_{n + 1} - {a}_{n} = \frac{{p}_{n}}{2} + \frac{{p}_{n + 1}}{2} \qquad ( 6 )$$

The quotient of the distance an of a detector element to the radiation fan and the corresponding height pₙ defines the ratio r:

$$r = \frac{{p}_{n}}{{a}_{n}} \qquad ( 7 )$$

This ratio must be constant in order to obtain a constant resolution of the scattering angle, i.e. it must be the same for all detector elements. The average distance of the detector elements can thus be recursively determined:

$${a}_{n + 1} = {a}_{n} ( \frac{1 + \frac{r}{2}}{1 - \frac{r}{2}} ) \qquad ( 8 )$$

It accordingly suffices to lay down the average distance of the first detector element from the radiation fan. The remaining average distances may be recursively calculated, or alternatively the remaining heights of the detector elements may be calculated when equation 7 is solved for aₙ and is substituted in equation 8 for an and aₙ₊₁.

FIG. 4 shows the dimensioning of such a detector by way of example. The lowermost detector element is 20 mm removed from the radiation fan. The distance d is 600 mm. A resolution of 5% is to be achieved, i.e. r=0.05. gₙ denotes the distance or pitch of the centers of two mutually adjoining detector elements.

It may happen that the sensitive regions of the detector elements do not immediately adjoin one another in the z-direction, but that a non-sensitive region, having a dimension s in the z-direction, is arranged between two mutually adjoining detector elements each time for technical reasons. Equation (8) then becomes:

$${a}_{n + 1} = \frac{{a}_{n} ( 1 + \frac{r}{2} ) + s}{1 - \frac{r}{2}} \qquad ( 9 )$$

FIG. 6 shows the dimensioning of such a detector by way of example. The lowermost detector element is 25 mm away from the radiation fan. The distance d is 1000 mm. A resolution of 4% is to be achieved, i.e. r=0.04. gₙ again denotes the distance between the centers or pitch of two mutually adjoining detector elements.

As in EP 1127546, a collimator arrangement 6 shown in FIG. 1a is present between the examination region 13 and the detector arrangement 16, comprising a plurality of planar lamellae 60. Said lamellae 60 are made of a material that strongly absorbs X-radiation and lie in planes which extend parallel to the axis of rotation 14 and intersect in the focus of the radiation source S. The collimator arrangement 6 accordingly subdivides the radiation fan 41 into a number of mutually adjoining sections, such that a column of detector elements can substantially be hit exclusively by primary or scattered radiation from a section.

The above explanations of FIG. 2 related to only a single scattering location. In actual fact, however, the X-rays are scattered in the entire object 13 along CF, so that each of the detector elements detects many scattered rays at various scattering angles. A data set consisting of several projections is acquired in order to be able to evaluate the above nevertheless separately. The object is rotated through a small angle relative to the radiation source and the detector for each projection, and the scattered rays are detected anew. The test data acquired by the detector 16 on the rotating gantry 1 of FIG. 1 are then supplied to an image processing unit 10 which will usually be present in a fixed location in the space and which is connected to the detector unit via a collector ring which operates in a contactless manner and is not shown in any detail.

The image processing unit 10 can carry out various image processing operations. Two reconstruction algorithms may be mentioned by way of example, which are suitable in particular for evaluating the data set mentioned above. A first algorithm is known from the German patent application with file no. DE10252662.1 (applicant's reference PHDE020257), not yet published, and a second one from the European patent application with file no. EP 03103789.8 (applicant's reference PHDE030349), not yet published. Since the algorithms are explained in great detail in both documents, a description thereof will be omitted here and instead reference is expressly made to the respective documents.

Alternatively to the detector of FIG. 2, a detector as shown in FIG. 1 may be provided in the computer tomograph of FIG. 1. The detector elements EL all have the same distance or pitch PIT with respect to one another in this detector DET. Furthermore, they all have the same height and thus the same effective dimensions. The required resolution is not achieved by the lower detector elements which lie close to the radiation fan not shown in FIG. 3, because the height is too great. To reduce the effective dimensions, absorption elements GD absorbing X-ray radiation are provided in front of these detector elements, which absorption elements are dimensioned such that the heights of the detector elements are reduced to values in accordance with what was explained above. No absorption elements are necessary for the upper detector elements, because the ratio r and thus the resolution is smaller than required. The absorption elements may also be constructed as a single component, which is then provided in the form of an absorption mask in front of the detector. The right-hand half shows the detector rotated through 90° about the z-axis, so that it can be recognized that the absorption elements are strip-shaped here.

FIG. 5 shows the dimensioning of such a detector by way of example. The average pitch of the detector elements is constant and has a value of 2.5 mm. The row with the lowermost detector element is 30 mm removed from the radiation fan. The distance d is 1000 mm. A resolution of 4% is to be achieved, so that r=0.04. It is visible for the upper detector elements that no absorption element is necessary here because r is below 4%.

The computer tomograph of FIG. 1 may also be operated in a second mode of operation. In this case only the attenuation of the primary radiation in the examination region is reconstructed. For this purpose, the first diaphragm arrangement 31 is removed from the radiation path, so that now only the second diaphragm arrangement 32 is active, generating a radiation cone 42. In addition, the collimator (not shown) is removed from the region between the detector arrangement 16 and the examination region 13. The detector and the absorption elements may also be removed, depending on the construction and size thereof. When test data are subsequently acquired, the gantry will rotate about the axis of rotation, so that all detector elements can be hit by primary radiation. The attenuation in a slice of the examination region is reconstructed in the subsequent reconstruction step. A suitable reconstruction method is described in the German patent application DE198451334 (applicant's reference PHD 98.123).

1. A computer tomograph for detecting rays that are elastically scattered in an object, wherein the object is present in an examination region and the scattered rays are scattered at different scattering angles, with a radiation source for permeating the examination region with primary radiation, and
a detector with detector elements which lie outside the region permeated by primary radiation and whose effective dimensions become smaller in the direction of decreasing scattering angles.

2. A computer tomograph as claimed in claim 1, with absorption elements which each cover a portion of a detector element such that the region of the scattering angle that can be detected by the respective detector element is reduced.

3. A computer tomograph as claimed in claim 1, with a polychromatic radiation source and with a detector having energy-resolving detector elements.

4. A computer tomograph as claimed in claim 1, with a radiation source for generating a fan-shaped ray and with absorption lamellae arranged between the detector and the object, which lamellae lie in planes that extend parallel to the axis of rotation and subdivide the radiation fan into sections such that the detector elements present in a column parallel to the rotation axis are substantially hit only by primary or scattered radiation from one and the same section.

5. A computer tomograph as claimed in claim 1,
with a radiation source for generating the primary radiation either in the form of a planar fan ray or a conical ray,
with a two-dimensional detector, and
with a first mode of operation in which a portion of the detector elements receives the scattered radiation generated by the planar fan ray, and with a second mode of operation in which the detector elements receive the primary radiation generated in the conical ray.

6. A detector for determining elastically scattered rays, which detector comprises at least one column comprising a plurality of energy-resolving detector elements, wherein the pitch of their centers and their dimensions increase towards a maximum value in the direction of the column.

7. A detector as claimed in claim 6, wherein the pitch of the centers of two mutually adjoining detector elements g is defined by g=aₙ₊₁−aₙ, and it holds that:
$${a}_{n + 1} = \frac{{a}_{n} ( 1 + \frac{r}{2} ) + s}{1 - \frac{r}{2}}$$
where r is a constant expressing the resolution of the scattering angle and s is the distance between two sensitive regions of mutually adjoining detector elements in the direction of the column.

8. A detector as claimed in claim 6, wherein pₙ=aₙ*r.

9. A detector as claimed in claim 6, comprising at least one detector element which is formed by a plurality of mutually adjoining sub-elements.
